(19) United States
(12) Patent Application Publication
(10) Pub. No.: US 2004/0093043 A1
(43) Pub. Date: May 13, 2004

(54) Irradiation device, particularly for carrying out photodynamic diagnosis or therapy

(76) Inventors: Susann Edel, Grunwald (DE); Georg Knott, Ponzberg (DE)

Correspondence Address: Aaron Wininger, Squire Sanders & Dempsey, 600 Hansen Way, Palo Alto, CA, 94304, US

(21) Appl. No.: 10/469,790

(22) PCT Filed: Apr. 18, 2001
(86) PCT No.: PCT/EP01/04409

(51) Int. Cl. A61N 5/06

(52) U.S. Cl. 607/88; 607/90; 607/91

(57) ABSTRACT

An irradiation device which is suitable, in particular, in the field of medicine for photodynamic diagnosis or therapy, comprises a plurality of flat luminous segments (1-4) which respectively generate radiation directed towards a particular irradiation surface (A). The individual luminous segments (1-4) are arranged at an angle to one another so that, when the irradiation device is arranged at a particular distance (D) from the irradiation surface (A), the directed radiations of the luminous segments (1-4) overlap essentially fully on this irradiation surface (A). In this way, homogeneous irradiation of the irradiation surface (A) with a high irradiation intensity will be achieved even in the event that each individual luminous segment (1-4) only has a relatively low irradiation intensity. The luminous segments (1-4) preferably have a multiplicity of uniformly distributed luminous means (6), in particular light-emitting diodes or laser diodes, in order to permit uniform irradiation of the irradiation surface (A).

The present invention relates to an irradiation device for achieving maximally homogeneous irradiation with a maximally high irradiation strength for various applications, in particular for medical, cosmetic or industrial applications.

In medicine, so-called photodynamic diagnosis (PDD) and photodynamic therapy (PDT) have been developed as important alternatives for the detection and treatment of neoplastic conditions of a patient in recent years. Both methods are based on the absorption of light with a suitable wavelength by a photosensitive substance, the so-called photosensitiser. If maximally selective concentration of the photosensitiser in the neoplastic tissue of the patient is achieved, then neoplastic conditions such as tumours can be detected relatively reliably with photodynamic diagnosis by evaluating the fluorescent light emitted as a result of the irradiation of the photosensitiser. A high tumour selectivity of the photosensitiser, on the one hand, and a high light absorption and quantum efficiency, on the other hand, are important when selecting the photosensitiser. Photodynamic therapy, conversely, utilises the fact that activation of molecular oxygen in the respectively treated tissue takes place between the photosensitiser and the biological environment via a complex interaction, the oxygen radicals generated in this way being highly reactive and capable of lethally damaging biomolecules in the immediate vicinity. In this way, again by concentration of a photosensitiser with subsequent irradiation, it is possible to achieve a therapeutic effect, for example the treatment of benign and malignant tumours, tissue lesions and/or warts, etc.

For the aforementioned medical photodynamic irradiation applications, irradiation devices have previously been known which have polychromatic irradiation sources with high-power lamps, these irradiation devices being usable, for example, for whole body irradiation or for irraddiating individual body sections, and for example a region of the oral cavity. In these conventional irradiation devices, besides the relatively complex structure, the use of the aforementioned high-power lamps is also problematic, since these high-power lamps on the one hand require relatively elaborate driving with a correspondingly high electricity consumption and, on the other hand, generate heat energy with broadband light which can be unpleasant for the patient, or even painful in the event of incorrect use.

For other applications, irradiation devices are in fact already known in which, for example, light-emitting diodes (LEDs) are arranged in the form of an array or a matrix in a plane (so-called LED cluster) as the luminous means. Light-emitting diodes, however, generally have a very low irradiation strength which is insufficient for intense irradiation as is required for the aforementioned photodynamic diagnosis and photodynamic therapy. Such irradiation devices, which have light-emitting diodes arranged close to one another in a conventional way, are therefore unsuitable for the medical application fields of photodynamic diagnostic and therapeutic use described above.

It is an object of the present invention to provide an irradiation device which, with the simplest possible means, makes it possible to irradiate an intended irradiation surface with a relatively high irradiation strength; in particular, it should be possible to ensure maximal irradiation homogeneity of the intended irradiation surface. The irradiation device should furthermore be suitable, in particular, for use in medical photodynamic diagnostic and therapeutic applications.

The above object is achieved according to the invention by an irradiation device with the features of claim 1. The dependent claims respectively define preferred and advantageous embodiments or applications of the present invention.

The irradiation device according to the invention comprises at least two flat luminous segments, which respectively generate directed radiation, the flat luminous segments being arranged at an angle to one another so that the individual directed radiations overlap essentially fully, or exactly, on an irradiation surface which lies at a particular distance from the irradiation device. In this way, it is possible to achieve an irradiation strength (mW/cm²) that is many times higher compared with conventional irradiation devices, one which allows intense irradiation in general, medical, cosmetic and industrial applications.

Regular arrangements of luminous means are preferably used as the luminous segments in this case, in particular light-emitting diodes or laser diodes, which emit for example UV radiation, IR radiation or visible light and generate the intended directed radiation of the respective luminous segments since the emission angle of the individual luminous means is limited to a particular value, in particular ≦30°.

The arrangement of luminous means, which normally have a relatively low irradiation strength, proposed in the scope of the present invention in the form of the aforementioned luminous segments, and the special geometrical arrangement of the individual luminous segments with respect to one another, make it possible to achieve a much higher irradiation strength compared with the prior art, the individual luminous means having a relatively low power consumption and not requiring elaborate driving. Owing to the use of luminous segments with a multiplicity of uniformly arranged luminous means, which may be arranged matricially in columns and rows or in mutually offset rows, a maximal degree of irradiation homogeneity is also ensured since the resultant exact optical imaging of the individual luminous segments on the intended irradiation surface leads to an optimum distribution of irradiation strength.

As already described above, commercially available light-emitting diodes may preferably be used as the luminous means, light-emitting diodes with a relatively small emission angle and a relatively high irradiation strength preferably being used. The choice of the luminous means which are respectively used will be determined, in terms of the primary emitted spectral wavelength, by the effective radiation wavelength required in the respective irradiation device. For example, light-emitting diodes with a wavelength in the vicinity of 630 nm, especially in the vicinity of 635 nm, may be used when, in the case of a medical photodynamic therapy application, the intended medical effect of this therapy occurs in the vicinity of this wavelength. For a medical photodynamic diagnosis application, wavelengths in the range 370 nm-430 nm may be necessary when the stimulation needed for the diagnosis occurs, for example, at the wavelengths 370 nm and 428 nm. For a cosmetic application, for example hair bleaching, luminous means may be used whose emitted wavelength lies in the vicinity of 500 nm, especially 502 nm, if the cosmetic hair bleaching effect occurs close to this wavelength. In order to avoid having to use a plurality of such irradiation devices for different applications, it is advantageous for the individual luminous segments of an irradiation device to have different groups of luminous means, each group of luminous means being arranged regularly distributed in the respective luminous segments and the individual groups of luminous means emitting different wavelengths, so that it is merely necessary for the corresponding group of luminous means to be activated, or switched on, as a function of the respectively intended application; a high irradiation strength with homogeneous irradiation can furthermore be achieved on the intended irradiation surface.

The irradiation strength multiplication generated on the intended irradiation surface is dependent on the number and the shape of the individual luminous segments, since each luminous segment is optically imaged onto the irradiation surface. The base surface of the individual luminous segments is then selected so that the radiations generated by the individual luminous segments overlap with a particular shape on the intended irradiation surface. If the individual luminous segments have a rectangular shape, then the shape of the overlap of the individual radiations is also rectangular. It is likewise possible to select polygonal or rounded shapes of the individual luminous segments, the base surface of the individual luminous segments not having to be identical. Rather, when selecting the base surface of the individual luminous segments, it is necessary to take into account that different radiation images of the individual luminous segments are obtained owing to the angled arrangement of the individual luminous segments with respect to one another, and the resultant differing alignment of the individual luminous segments relative to the intended irradiation surface, that is to say the shape of a luminous segment perpendicularly facing the irradiation surface will be imaged without modification, whereas the shape of a luminous segment arranged at a relatively acute angle to the irradiation surface will be imaged with distortion. If, for example, a circular overlap of the radiations of the individual luminous segments is to be achieved on the irradiation surface, it may therefore be necessary to design the luminous segments arranged at a relatively acute angle to the irradiation surface with an elliptical shape, whereas the luminous segments arranged at a less acute angle to the irradiation surface, or at right angles, may be designed with a circularly rounded shape so that circular-shaped beam shapes respectively overlap on the intended irradiation surface.

The individual luminous segments, or luminous segment supports, may be arranged adjacent or next to one another, and they may be fitted in a common housing. In particular for irradiating individual body regions of a living being, or of a patient, it is then expedient for the individual luminous segments to be fitted in a portable housing, or in a handpiece, so that the irradiation device according to the invention may, for example, also be used for irradiating a region of the oral cavity of a patient.

Various exemplary embodiments of an irradiation device according to the invention and of an irradiation system according to the invention, which, for example, are suitable for medical photodynamic diagnosis or therapy, will be explained below with reference to the appended drawing; in particular, irradiation devices and irradiation systems are proposed which can be used for irradiating body regions or for whole body irradiation. The invention may also be employed for irradiating skin surfaces, for example for treating acne, without prior administration of photosensitisers since the high irradiation strength is sufficient for treating acne, and the bacteria which cause acne themselves produce protoporphyrin. The present invention is moreover suitable for cosmetic use, for example for hair bleaching or depilation, or for industrial applications, for example for coupling light into an optical waveguide (preferably with the aid of a converging lens) or for curing (the all-round coating or encoding of a cable, for example). The present invention may also be used for all-round irradiation of transparent small tubes or capillaries with liquids, for example blood, or cell cultures etc, contained in them. The present invention is not, of course, restricted to the application fields described in detail below; rather, it may be employed in general wherever the intention is to achieve, by relatively simple means, a relatively high irradiation strength with an especially homogeneous irradiation strength distribution on an intended irradiation surface.

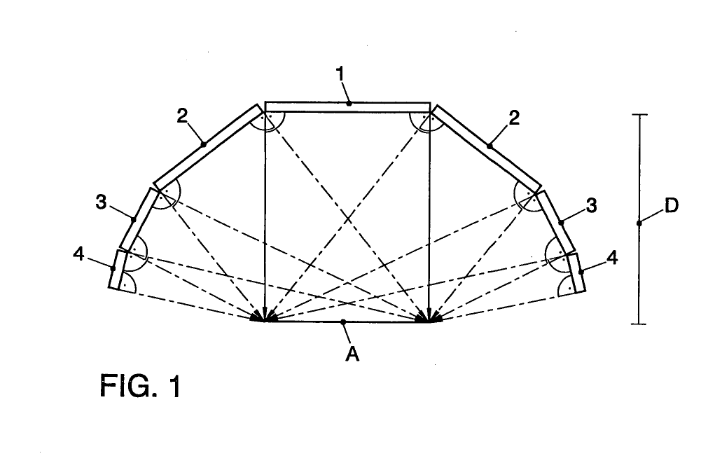

FIG. 1 shows a cross-sectional view of an irradiation device according to a first exemplary embodiment of the present invention,

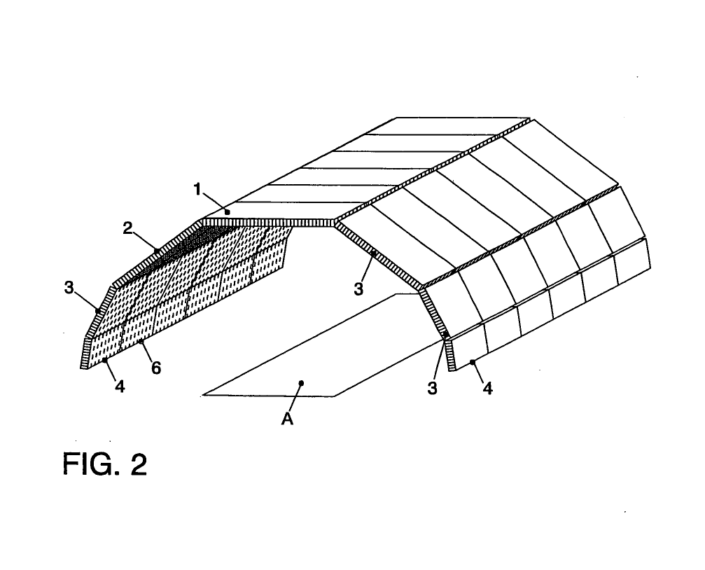

FIG. 2 shows a perspective view of an irradiation device according to a second exemplary embodiment of the present invention,

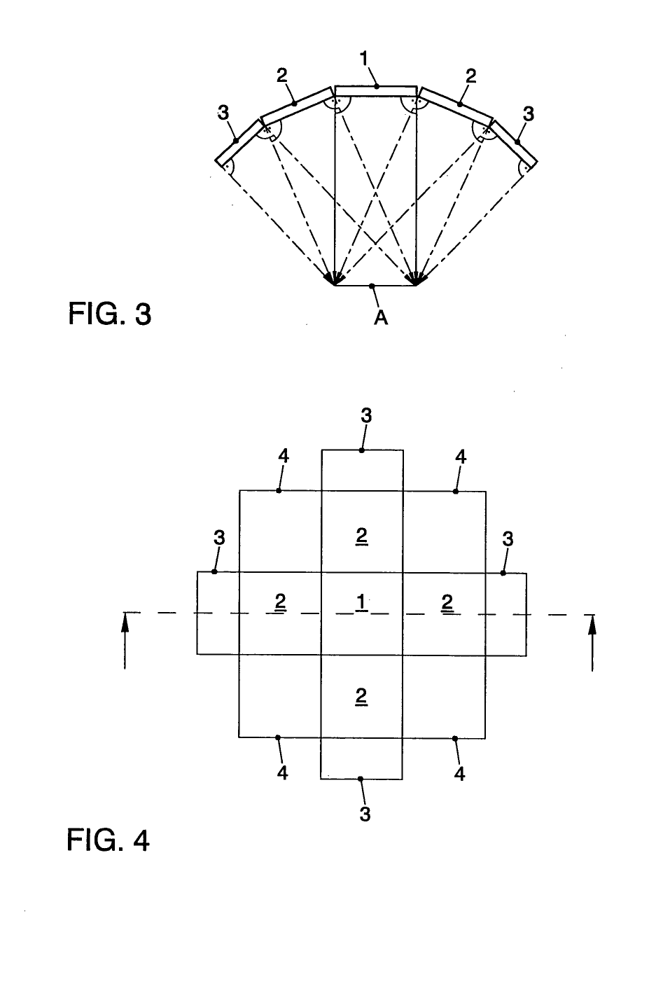

FIG. 3 shows a cross-sectional view of an irradiation device according to a third exemplary embodiment of the present invention,

FIG. 4 shows in plan view the arrangement of the individual luminous segments of the exemplary embodiment represented in FIG. 3,

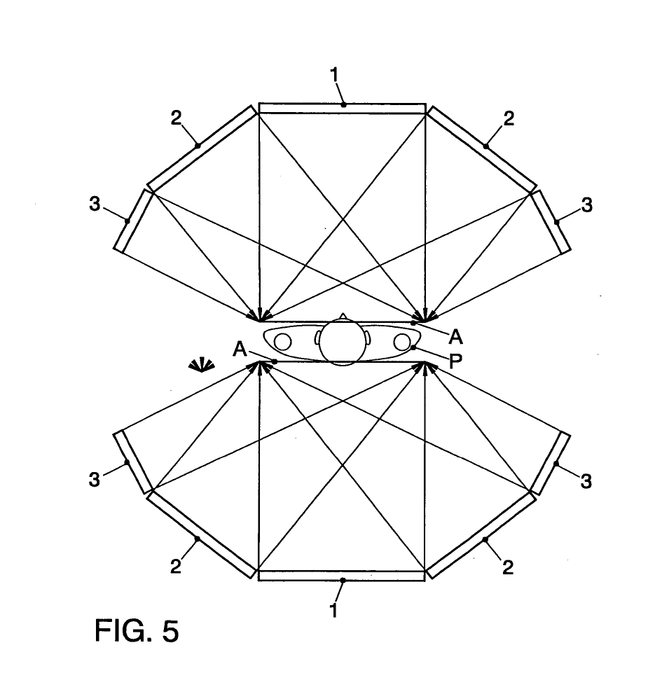

FIG. 5 shows an irradiation system with two irradiation devices according to a fourth exemplary embodiment of the present invention,

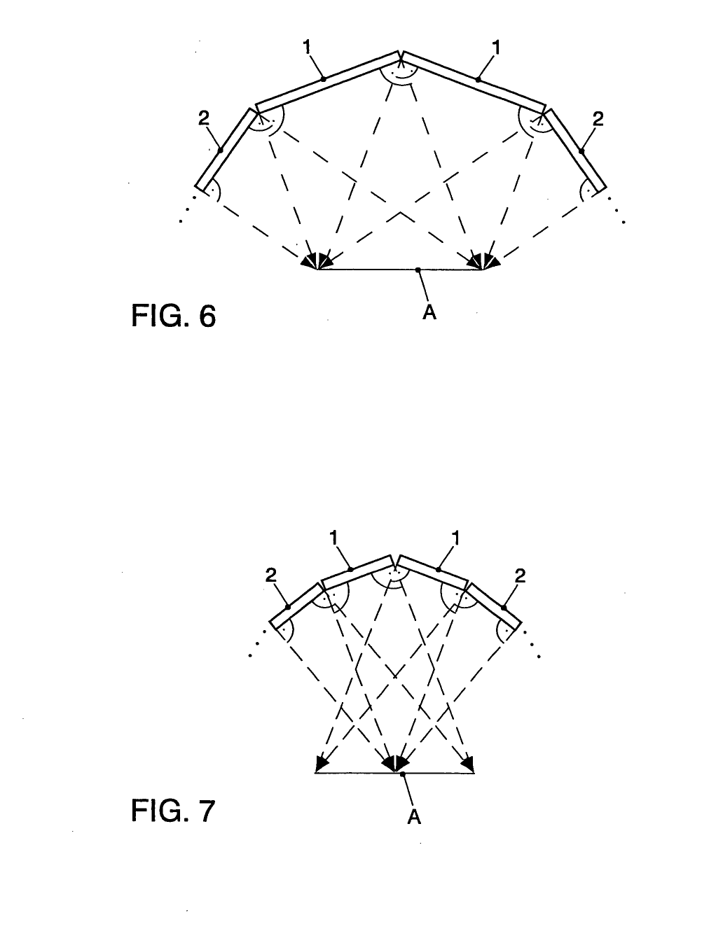

FIG. 6 shows a cross-sectional view of an irradiation device according to a fifth exemplary embodiment of the present invention,

FIG. 7 shows a cross-sectional view of an irradiation device according to a sixth exemplary embodiment of the present invention,

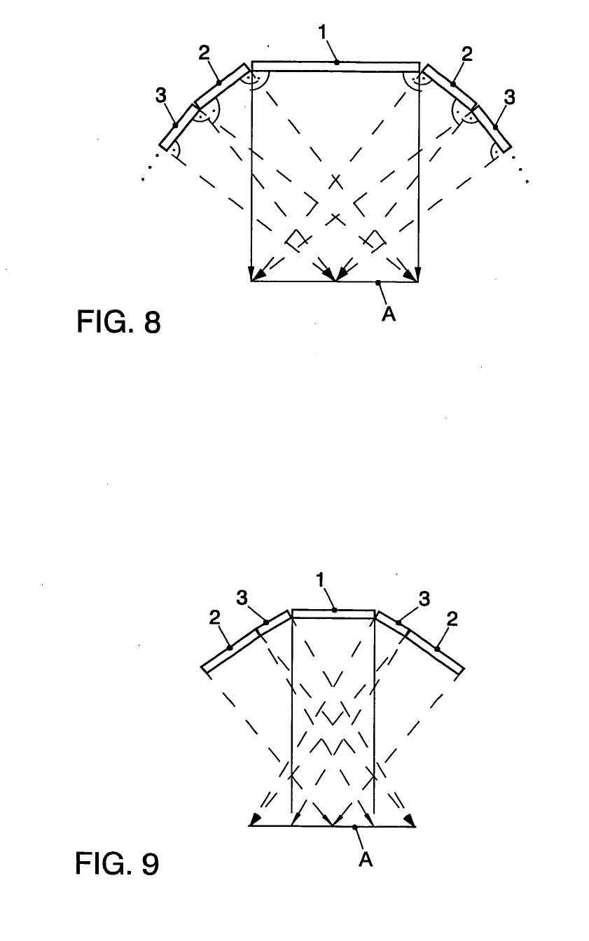

FIG. 8 shows a cross-sectional view of an irradiation device according to a seventh exemplary embodiment of the present invention,

FIG. 9 shows a cross-sectional view of an irradiation device according to a eighth exemplary embodiment of the present invention,

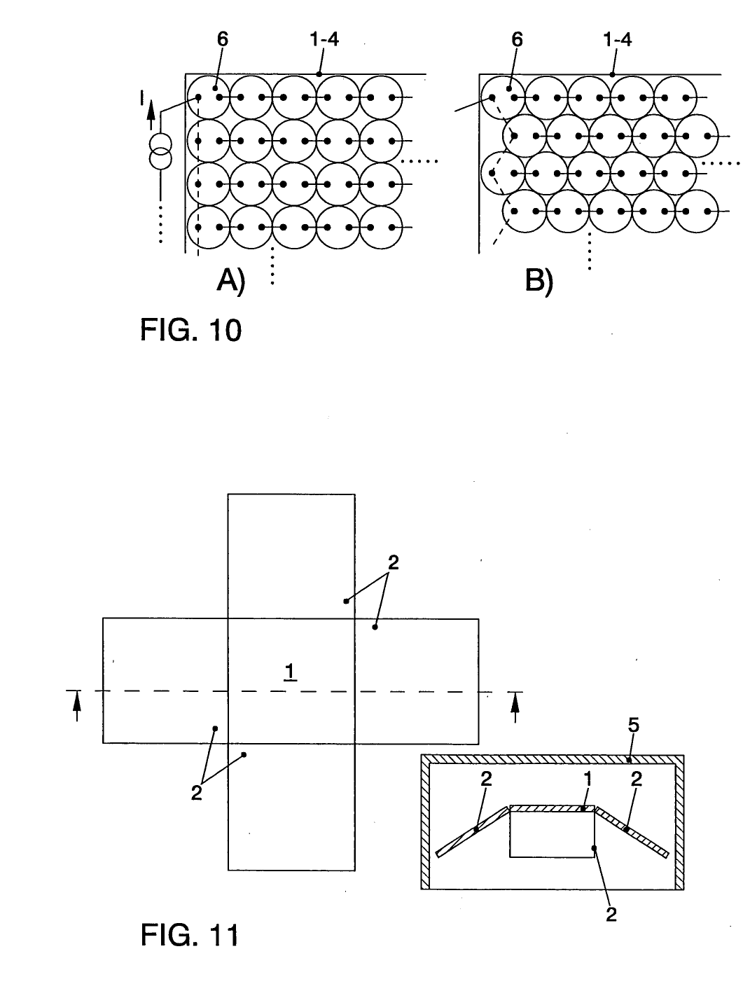

FIG. 10A and FIG. 10B show examples of a regular arrangement of light-emitting diodes in a luminous segment according to the present invention,

FIG. 11 shows representations of an irradiation device according to a ninth exemplary embodiment of the present invention,

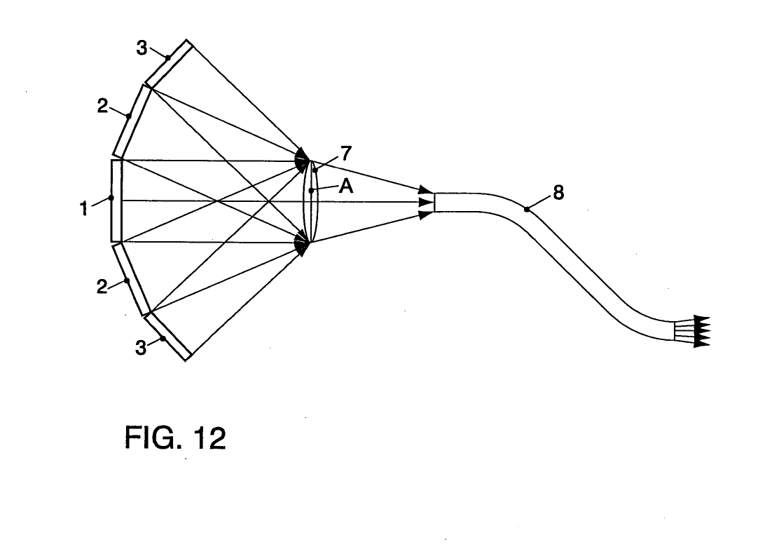

FIG. 12 shows the use of an irradiation device according to the invention for coupling light into an optical waveguide,

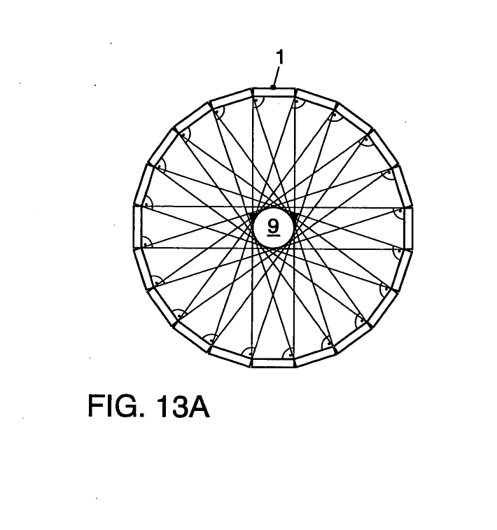

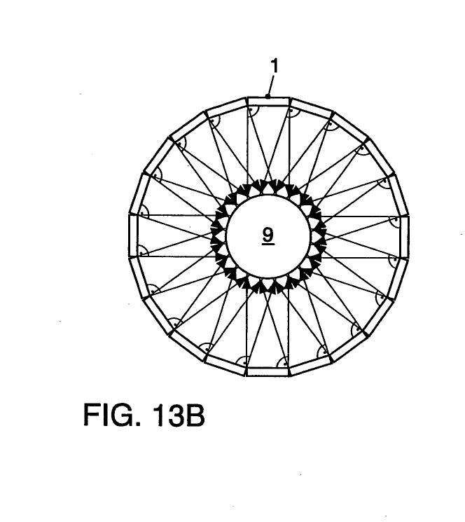

FIG. 13A and FIG. 13B show examples of the use of an irradiation device according to the invention for all-round irradiation of an elongate medium, for example to cure the all-round coating or encoding of a cable or to irradiate a transparent small tube or a transparent capillary with a biological material (for example blood or a liquid with cell cultures) contained in it,

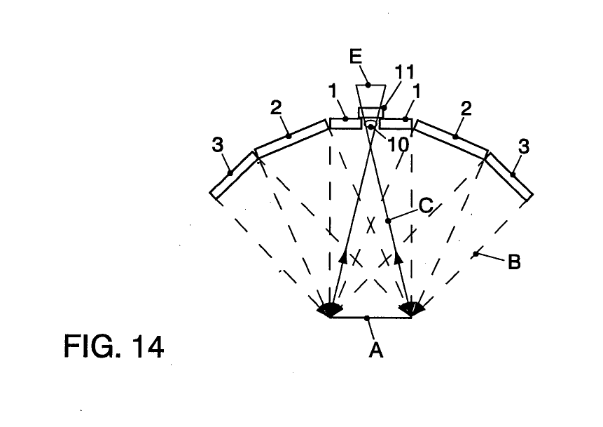

FIG. 14 shows an irradiation device according to the invention, which is constructed similarly to the irradiation device shown in FIG. 3, for allowing photodynamic diagnosis (PDD),

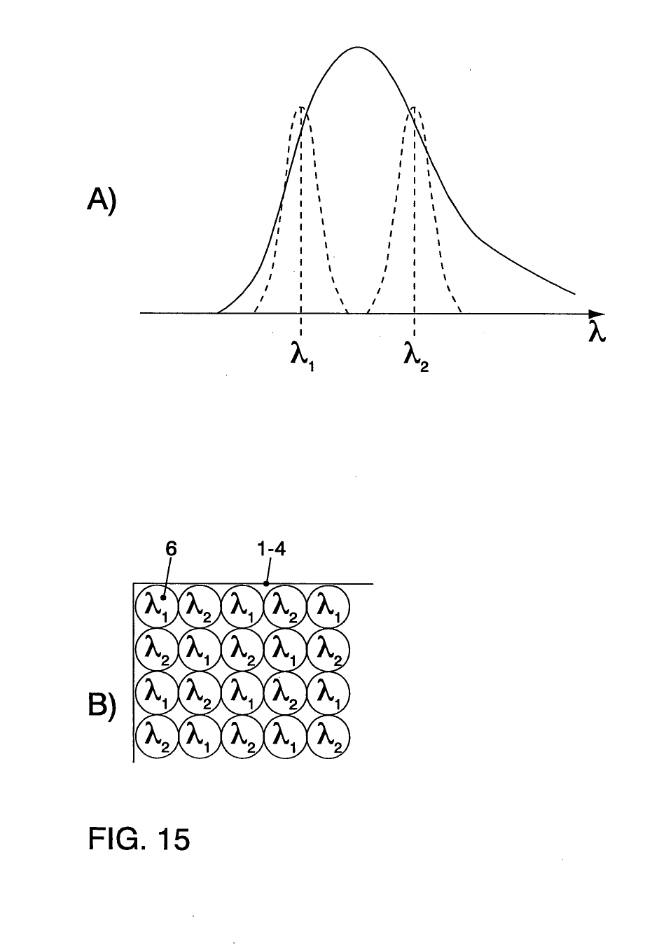

FIG. 15A and FIG. 15B show representations to explain the arrangement of luminous means, for example light-emitting diodes, of differing wavelength on a luminous segment support of an irradiation device according to the invention, and

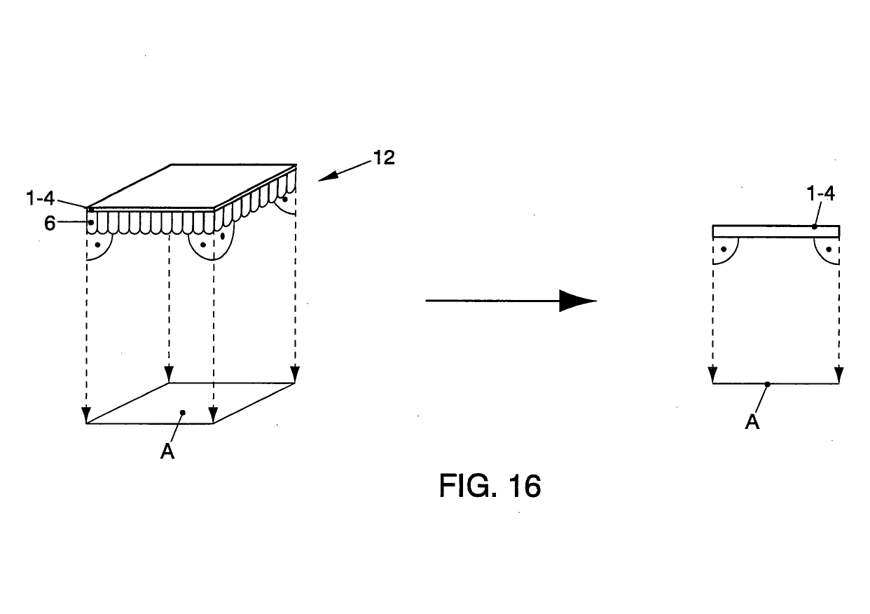

FIG. 16 shows a representation to clarify the simplified form of representation chosen in the appended figures for the luminous segment supports of the irradiation device according to the invention.

FIG. 1 represents a cross-sectional view of an irradiation device according to the invention, a plurality of luminous segment supports with corresponding luminous segments 1-4 being arranged at an angle to one another. Each luminous segment 1-4 is in this case preferably formed by a regular arrangement of suitable luminous means, in particular light-emitting diodes or laser diodes.

As can be seen from FIG. 1, the individual luminous segments 1-4 respectively generate directed radiation, it being assumed as an approximation that the individual luminous segments 1-4 emit perpendicularly, as represented in FIG. 1. The luminous segments 1-4 are arranged adjacent or next to one another, so that almost exact or essentially full superposition of the individual radiations takes place when the irradiation device is brought close to an intended irradiation surface. In this way, it is possible to achieve a significant increase in the irradiation strength, even though the luminous means being used are, for example, light-emitting diodes or laser diodes which have a significantly reduced irradiation strength compared with high-power lamps. The distance D, between the irradiation surface A and the irradiation device, at which the focusing of the individual radiations as described above takes place, and which is defined according to FIG. 1 by the distance between the irradiation surface A and the luminous segment 1 immediately opposite it, essentially depends in this case on the angled arrangement of the individual luminous segments 1-4.

As has already been mentioned, the individual luminous segments 1-4 have a rectangular base surface in the exemplary embodiment which is represented, the base surface of the individual luminous segments 1-4 being imaged onto the irradiation surface A so that the individual radiations of the luminous segments 1-4 also overlap in a region with a rectangular base surface on the irradiation surface A. When choosing the base surface of the individual luminous segments 1-4, it should be borne in mind that the base surface of the luminous segment 1 which is arranged immediately perpendicularly opposite the irradiation surface A is imaged onto the irradiation surface A without modification, whereas the luminous segments 2-4 arranged at an angle to the irradiation surface A are imaged onto the irradiation surface A with a greater or lesser degree of distortion. The more acute the angle between the irradiation surface A and the normal taken from the luminous segments 2-4, the smaller the base surface of the respective luminous segment 2-4 can be chosen to be, since the distortion of the image of the respective luminous segment increases.

Apart from the width, however, the dimensions of the individual luminous segments 1-4, that is to say the height or thickness and depth, may be chosen to be identical.

In order to clarify the form of representation chosen in FIG. 1 and in the other figures for the individual luminous segment supports, or luminous segments 1-4, FIG. 16 represents a perspective view of an example of such a luminous segment support 12 according to the invention. As can be seen from the representation on the left in FIG. 16, the luminous segment support 12 comprises a multiplicity of luminous means 6 distributed uniformly over its surface, for example light-emitting diodes or laser diodes, which generate directed radiation so that the radiation generated overall by the resultant luminous segment 14 is imaged essentially perpendicularly onto the irradiation surface A. In this way, the radiation imaged onto the irradiation surface A has a base surface which depends on the base surface of the respective luminous segment 1-4, that is to say on the base surface of the arrangement of the individual luminous means 6. In the example represented in FIG. 16, the irradiation surface A is arranged parallel to the luminous segment support 12, or the corresponding luminous segment 1-4, so that the shape of the irradiation surface A corresponds to the shape of the respective luminous segment 1-4. As shown in the representation on the right in FIG. 16, the appended figures do not in general show a separate representation of a luminous segment support with the individual luminous means located on it; rather, merely the luminous segment 1-4 corresponding to the arrangement of the luminous means 6 on the luminous segment support 12 is represented for simplicity, as is shown in the representation on the right in FIG. 16, for example, in a cross-sectional view.

FIG. 2 shows an irradiation system which, for example, may be used for whole body irradiation of a patient for medical photodynamic therapy (PDT), and which is based on the structure represented in FIG. 1. From FIG. 2, it can be seen in particular that a plurality of groups of luminous segments 14 are arranged adjacent or next to one another in the longitudinal direction of the illumination system so that, overall, a hemicylindrical tube is formed on the inner surface of which the individual luminous segments are arranged, respectively with a multiplicity of luminous means 6. The radiation emerging from the individual luminous segments 14 is hence directed into the interior of this hemicylindrical tube, so that a relatively large irradiation surface A, whose longitudinal extent corresponds to the length of the irradiation system which is represented, can be irradiated homogeneously and with a high irradiation strength.

It can be also seen from FIG. 2 that the depth of the individual luminous segments 1-4, that is to say the dimension in the longitudinal direction of the hemicylindrical tube which is represented, is identical; the mid-axes of the luminous segments 1-4 extending perpendicularly to the longitudinal direction of the hemicylindrical tube being aligned with one another so that the luminous segments 1-4 of a group of luminous segments extend close together.

FIG. 3 represents a further exemplary embodiment of an irradiation device according to the invention in a cross-sectional view; in contrast to FIG. 1, only five luminous segments 1-3 are used, the radiations of which overlap fully, that is to say exactly, on the irradiation surface A. In this case, the irradiation device represented in FIG. 3 may in particular be part of an irradiation system whose luminous segments projected into a plane, that is to say folded into the plane of the luminous segment 1, may have the arrangement and shape represented in FIG. 4, the cross-sectional view represented in FIG. 3 having been taken in the arrow direction along a section line represented by dashes in FIG. 4.

As can be seen from FIG. 4, the luminous segments 1-3 represented in a cross-sectional view in FIG. 3 are arranged in a row; perpendicularly to this row, the luminous segment 1 is arranged next to two further luminous segments 2, which are arranged at the same angle as the two aforementioned luminous segments 2 with respect to the luminous segment 1. Likewise, in addition to the luminous segments 3 represented in FIG. 3, two further luminous segments 3 are arranged at the end, all of which are arranged angled at the same angle with respect to the luminous segment 1. Between two luminous segments 2 arranged diagonally next to one another, a further luminous segment 4 is respectively provided, and is arranged at an angle to the other luminous segment 1-3 so that the radiations output by the individual luminous segments 1-4 overlap essentially fully on the intended irradiation surface A, so that a maximum irradiation intensity with a homogeneous irradiation intensity distribution occurs on the irradiation surface A.

FIG. 14 represents an irradiation device according to the invention for medical photodynamic diagnosis (PDD), the basic structure of the irradiation device shown in FIG. 14 essentially corresponding to the basic structure of the irradiation device shown in FIG. 3. In the irradiation device shown in FIG. 14, five luminous segments 1-3 are again used, the radiations of which overlap fully, that is to say exactly, on the irradiation surface A. The luminous segments of the irradiation device may, folded into the plane of the luminous segment 1, have the arrangement and shape represented in FIG. 4. If the irradiation device is positioned so that the irradiation surface A lies on an intended body section, fluorescent radiation C is induced as a function of the photosensitive substance administered to the patient, i.e. the photosensitiser, by the radiations B of the individual luminous segments 1-3 which overlap on the irradiation surface A, and this fluorescent radiation C is emitted by the irradiation surface A. In a PDD application, the wavelength of the radiations generated by the individual luminous segments 1-3 may, for example, lie in the range between 370 nm and 430 nm. In central luminous segment 1 there is an opening, preferably with a lens 10 with the aid of which the emitted fluorescent radiation C is focused and imaged onto an observation plane E. An optical filter 11 is also provided, which filters out the intended radiation, for example porphyrin fluorescent radiation in the wavelength range 600 nm-750 nm, from the radiation detected by the lens 10, so that the external interferences (for example due to external radiation sources) can be eliminated. Via the observation plane E, the fluorescent radiation thus stimulated and detected can be observed and evaluated, for example with the eye or a suitable optical evaluation system (for example a camera lens), so as to identify neoplastic conditions, for example tumours, on the skin surface being observed.

FIG. 5 shows an irradiation system according to the invention with two irradiation devices of the type represented in FIG. 1, although in contrast to FIG. 1, the two irradiation devices respectively comprise only five luminous segments 1-3. As can be seen from FIG. 5, the irradiation system represented in FIG. 5, which is constructed similarly to FIG. 2 as viewed in perspective, allows whole body irradiation of a standing patient P who needs to be positioned in the vicinity of the irradiation surfaces A of the two irradiation devices, the radiations of the luminous segments 1-3 of the upper irradiation device fully overlapping on the upper irradiation surface A, and the radiations of the luminous segments 1-3 of the lower irradiation device fully overlapping on the lower irradiation surface A. Of course, the irradiation system shown in FIG. 5 is also suitable for whole body irradiation of a recumbent patient P, if the patient P is lying in the vicinity of the lower irradiation surface A on a radiation-transmissive table, for example made of quartz glass or acrylic glass.

FIG. 6 represents a further exemplary embodiment of an irradiation device according to the invention, two luminous segments 1 and two luminous segments 2 respectively being provided and being arranged at an angle to one another so that the radiation which is generated by the individual luminous segments 1, 2, and which is emitted essentially perpendicularly, overlaps in the vicinity of the irradiation surface A. In contrast to the exemplary embodiments explained above, no luminous segment is arranged parallel to the irradiation surface A in the exemplary embodiment represented in FIG. 6; rather, all the luminous segments 1, 2 are arranged at a certain angle to the irradiation surface A in the cross-sectional plane which is represented.

FIG. 7 represents a further exemplary embodiment, which is configured similarly to the exemplary embodiment represented in FIG. 6, although the arrangement of the individual luminous segments 1, 2 is selected so that only the radiations generated by two luminous segments 1, 2 next to one another respectively overlap, that is to say the radiations generated by the two left-hand luminous segments 1, 2 together light a right-hand section of the irradiation surface A, whereas the radiations generated by the two right-hand luminous segments 1, 2 illuminate a left-hand section of the irradiation surface A, so that each region of the irradiation surface A is irradiated by two luminous segments 1, 2. This exemplary embodiment hence also provides homogeneous illumination of the irradiation surface A with a relatively high irradiation intensity.

FIG. 8 represents a further exemplary embodiment, in which a luminous segment arranged parallel to the irradiation surface A illuminates the entire irradiation surface A, whereas two luminous segments 2 and 3 arranged at an angle to the luminous segment 1 respectively illuminate only the left-hand or right-hand part of the irradiation surface A, so that, overall, each region of the irradiation surface A is irradiated by three luminous segments. This exemplary embodiment hence also provides irradiation of the irradiation surface A with a high intensity and a homogeneous irradiation strength distribution.

A luminous segment 1 is likewise arranged parallel to the irradiation surface A in the exemplary embodiment represented in FIG. 9, and two luminous segments 2 arranged at an angle to the luminous segment 1 respectively illuminate half of the section irradiated by the luminous segment 1. In addition, two further luminous segments 3 arranged at an angle are provided, which illuminate those sections of the irradiation surface A not being irradiated by the luminous segment 1, so that each region of the irradiation surface A is respectively irradiated by two luminous segments in this exemplary embodiment as well. This exemplary embodiment hence also provides homogeneous irradiation of the irradiation surface A with a high irradiation intensity, so long as the irradiation intensities of the individual luminous segments 1-3 have been selected accordingly. Depending on the choice of the irradiation intensity of the individual luminous segments 1-3, a defined reduction or increase of the irradiation intensity may alternatively also be achieved in subregions of the irradiation surface A, for example in the edge regions irradiated by the luminous segments 3.

The individual luminous segments 1, 2 of a further exemplary embodiment are represented in FIG. 11, in a similar way to the form of representation chosen in FIG. 4, and a cross-sectional view of the angled arrangement of these luminous segments along a section line shown represented by dashes is also shown.

As can be seen from FIG. 11, a central luminous segment 1 is provided and star-shaped luminous segments 2 are arranged next to it. The luminous segments 2 are respectively angled off downwards from the luminous segment 1 by the same angle, as can be seen from the cross-sectional view represented in FIG. 11. The luminous segments 1, 2 are, in particular, arranged so that their base surface is imaged onto a common irradiation surface which, in the exemplary embodiment which is represented, is arranged parallel to and below the luminous segment 1. It is furthermore indicated in FIG. 11 that all the luminous segments 1, 2 may be located in a common housing 5 which has an exit opening at its bottom for the radiations generated by the individual luminous segments 1, 2. The housing 5 may, in particular, be the housing of a handpiece so that, for example, it is possible to irradiate an oral cavity section of a patient with a miniaturised design of the luminous segments 1, 2. When a larger (static) housing 5 is used, for example, it is possible to irradiate the entire field of view of a patient.

As has already been mentioned, a multiplicity of luminous means are preferably used in each case for the individual luminous segments 14 of the exemplary embodiments described above, these being arranged uniformly on the individual luminous segments 1-4. Corresponding exemplary embodiments are represented in FIG. 10A and FIG. 10B.

In particular, narrow-emitting light-emitting diodes or laser diodes with the highest possible irradiation intensity are preferably suitable as the luminous means 6 in this case. The advantage of using light-emitting diodes or laser diodes is that they generate an advantageously selective narrowband emission spectrum, which can be used for therapeutic/diagnostic irradiations with the highest possible selectivity in particular spectral ranges, which depend on the photosensitiser respectively being used. If the intended treatment spectrum is to be a broadband spectrum, this can be achieved by using different luminous means, which emit for example in the UV range, IR range or in the visible light range, inside a luminous segment (cf. FIG. 15A for the generation of an optimum PDD spectrum for fluorescence stimulation or, for example in a PDT application, the use of a narrowband spectrum without thermal light components). The emission angle of the selected light-emitting diodes or laser diodes should be as narrow as possible; when irradiating larger irradiation surfaces, the emission angle is less critical than in the case of smaller irradiation surfaces. Furthermore, the maximum emission angle of the light-emitting or laser diodes depends on the distance D (cf. FIG. 1) from the irradiation surface. Trials have shown that a maximum irradiation angle of 30°, preferably 20°, should be kept to, although light-emitting or laser diodes with an emission angle of 6° or even 3° may ideally also be employed. The use of narrow-emitting light-emitting diodes or laser diodes is advantageous since, for carrying out the present invention, it is necessary for directed radiations to be generated by the individual luminous segments, since only in this case is exact overlap of the individual radiations on the intended irradiation surface, and therefore a homogeneous irradiation of the irradiation surface with a high irradiation intensity, guaranteed.

As shown in FIG. 10A, light-emitting diodes 6 which are arranged matricially in rows and columns may, for example, be used for the luminous segments 14. A uniform arrangement of the light-emitting diodes on the individual luminous segments 1-4 is advantageous in order to achieve homogeneous irradiation of the intended irradiation surface. Instead of a matricial arrangement of the individual light-emitting diodes, the light-emitting diodes 6 may also be arranged in rows according to FIG. 10B, the rows being arranged alternately offset with respect to one another.

The individual light-emitting diodes (or luminous means) 6 of the same luminous segment 1-4 preferably emit light with the same wavelength spectrum.

Likewise, however, it is also conceivable for a plurality of groups of light-emitting diodes 6 to be provided on the individual luminous segments 1-4, or the corresponding printed-circuit boards, the light-emitting diodes within the individual groups respectively being distributed uniformly over the corresponding luminous segment, and the light-emitting diodes 6 of the individual groups emitting light with a different wavelength, so that the same luminous segment can be used to emit light with different wavelengths by selectively driving or activating the light-emitting diodes. An example of such an arrangement of light-emitting diodes 6, which are subdivided into two groups with a different wavelength or different wavelength spectra, is represented in FIG. 15B. As can be seen from FIG. 15B, the light-emitting diodes of one light-emitting diode group generate radiation in the vicinity of a primary wavelength λ₁, whereas the light-emitting diodes of the other light-emitting diode group generate radiation in the vicinity of a primary wavelength λ₂. The light-emitting diodes of a light-emitting diode group are respectively distributed uniformly over the corresponding luminous segment 1-4 so as to obtain, overall, the uniform arrangement of light-emitting diodes 6 shown in FIG. 15B which emit light with different wavelength spectra, that is to say a light-emitting diode 6 with the primary wavelength λ₁ and a light-emitting diode 6 with the primary wavelength 2 are respectively arranged alternately on the luminous segment 1-4.

As shown in FIG. 15A, radiation with the primary wavelength λ₁ or with the primary wavelength 2 will be generated as a function of the activation either of the light-emitting diodes of the first light-emitting diode group or of the light-emitting diodes of the second light-emitting diode group. When all the light-emitting diodes of both light-emitting groups are activated simultaneously, an overall spectrum which most closely approximates the desired spectrum, and which is shown in FIG. 15A, is obtained for the radiation emitted by the respective luminous segment 1-4. This arrangement may, for example, be selected whenever no luminous means exists for the desired spectrum and a spectrum most closely approximating the desired spectrum has to be achieved by using a plurality of different luminous means.

The use of light-emitting diode groups with different wavelength spectra on the same luminous segment 14 is advantageous, in particular, whenever the irradiation device is, for example, intended to be used for medical photodynamic therapy applications with different photosensitisers. Since these photosensitisers usually react at different wavelengths, these spectral differences can be accommodated by corresponding activation of the respective light-emitting diode group. The same applies to medical photodynamic diagnosis applications; in this way, it is possible to employ different stimulation spectra according to the type of fluorescent substance.

As indicated in FIG. 10A, the individual light-emitting diodes 6 are preferably operated with a constant current I from a constant-current source; a plurality of light-emitting diodes 6, for example 16 light-emitting diodes, may respectively be connected in series. Driving the individual light-emitting diodes 6 with a constant current is advantageous since a more uniform brightness of the light-emitting diodes 6 of the same luminous segment 1-4 can be achieved in this way. When a constant current is used, the brightness of the individual light-emitting diodes 6 can furthermore be controlled more easily. A common constant-current source (parallel operation) may be provided for the individual light-emitting diode rows. Likewise, however, it is also possible to use separate constant-current sources for each light-emitting diode row or other groupings of light-emitting diodes.

The preferred application field of medical photodynamic diagnosis or therapy, in particular, has been described with the aid of the exemplary embodiments described above. For carrying out photodynamic therapy, the luminous segments used in the respective irradiation device may, in particular, be configured so that they emit radiation in the red range, in particular in the range around 635 nm. For photodynamic diagnosis, however, stimulation radiation in the UV or near-UV range is generally suitable, in which case the wavelength should lie especially between 370 nm and 430 nm. When using the principle, represented in FIG. 15A and FIG. 15B, of luminous means or light-emitting diodes with different wavelength spectra, it is hence possible to select λ₁=370 nm and λ₂=430 nm, for example.

Many other application fields besides the aforementioned application areas are moreover conceivable for the present invention, and these may, for example, be found in the cosmetic or industrial sectors. For example, the irradiation device according to the invention, or the irradiation system according to the invention, may also be used for cosmetic purposes for hair bleaching or depilation; to this end, for example, wavelengths in the visible range (VIS range) may be employed, especially in the range around 500 nm. Wavelengths >800 nm (IR diodes) or <380 nm (UV diodes) may likewise be used.

Further preferred application areas will be explained below with reference to FIG. 12 and FIG. 13A, as well as FIG. 13B.

FIG. 12 shows an irradiation device of the type represented in FIG. 3, with luminous segments 1-3 which are arranged at an angle so that the radiations emitted perpendicularly by the individual luminous segments 1-3 are concentrated, or focused, on an irradiation surface A, that is to say the radiations emitted by the individual luminous segments 1-3 overlap fully on this irradiation surface A. The arrangement is selected so that the irradiation surface A lies on or in a converging lens 7, through which the incident radiation is coupled into an optical waveguide 8. The advantage of the arrangement shown in FIG. 12 is hence that, owing to the overlap of the radiations generated by the individual luminous segments 1-3, light with a high irradiation intensity can be generated in a straightforward way for the coupling into an optical waveguide 8. The converging lens 7 may also be obviated in the event that the radiations of the luminous segments 1-3 are focused onto the light entry face of the optical waveguide 8.

In the application case represented in FIG. 13A, a multiplicity of identically constructed luminous segments 1 are arranged so that they form a circularly rounded shape in cross section. Each luminous segment 1 generates radiation which has the same diameter and is essentially emitted perpendicularly. In this way, a region around the mid-point of the cross-sectional shape shown in FIG. 13A is irradiated uniformly by all the luminous segments 1, so that there is irradiation with a maximum and uniformly distributed irradiation intensity in this region.

The arrangement shown in FIG. 13A may, for example, be used for curing a body 9 arranged in this overlap region of the radiations of the individual luminous segments 1, or the surface of this body. If the individual luminous segments 1 have a corresponding length, or if a plurality of the irradiation devices shown in FIG. 13A are arranged next to one another in the longitudinal direction so that a cylindrical tube is formed by the luminous segments 1, then, for example, the coating or encoding of a cable 9 arranged along the mid-axis of the tube can be cured with this arrangement. The body 9 which is being irradiated by the individual luminous segments 1, and which is arranged in the overlap region of the radiations of the individual luminous segments 1, may, for example, also be a body made of a light-transmissive or transparent material which extends in the longitudinal direction of the irradiation device, for example a small tube or a capillary, so that liquids (for example blood) contained in it, or cells or substances being transported in liquids contained in it, can be stimulated, illuminated or represented (marked) by the light. In this way, for example, it is also possible to carry out photodynamic diagnosis or photodynamic therapy of individual blood cells, or other cell cultures, which are being transported in liquids.

In the example shown in FIG. 13A, the diameter of the radiations generated by the individual luminous segments 1 corresponds essentially to the cross-sectional diameter of the body 9 arranged in the overlap region of the individual radiations. However, it is of course also conceivable for the arrangement of the luminous segments 1 and the diameter of the radiations generated by the individual luminous segments 1 to be selected so that the diameter of the radiations is in each case smaller than the diameter of the body 9 to be irradiated all-round, and so that the individual radiations overlap uniformly on the surface of the body 9 to be irradiated, as represented in FIG. 13B. In particular, the arrangement is such that each surface point of the body 9 is irradiated by at least two luminous segments 1, or even three of them as in the exemplary embodiment which is represented. In other regards, the explanations pertaining to FIG. 13A apply similarly to the irradiation device shown in FIG. 13B.

1. Irradiation device,
with at least two luminous segment supports (12), each luminous segment support having a flat luminous segment (1-4) with a multiplicity of uniformly arranged luminous means (6), the luminous segment supports (12) being arranged at an angle to one another,
characterised in that
the emission angle of the individual luminous means (6) is at most 30°, so that directed and essentially perpendicularly emitted radiation is generated by each luminous segment (1-4), and
the luminous segment supports (12) are arranged at an angle to one another so that, when the irradiation device is arranged at a particular distance (D) from an irradiation surface (A), the radiations emitted essentially perpendicularly by the individual luminous segments (1-4) overlap essentially fully on this irradiation surface (A).

2. Irradiation device according to claim 1,
characterised in that
the luminous means (6) are arranged uniformly in columns and rows on the individual luminous segments (1-4).

3. Irradiation device according to claim 1,
characterised in that
the luminous means (6) in the individual luminous segments (1-4) are arranged in rows which are alternately offset with respect to one another.

4. Irradiation device according to any one of the preceding claims,
characterised in that
a power supply unit is provided for supplying the individual luminous means (6) of the luminous segments (1-4) with constant current.

5. Irradiation device according to claim 4 and claim 2 or 3,
characterised in that
the luminous means (6) respectively arranged in a row are connected in series and connected to the power supply unit.

6. Irradiation device according to any one of the preceding claims,
characterised in that
the luminous means (6) are light-emitting diodes.

7. Irradiation device according to any one of claims 1-5,
characterised in that
the luminous means (6) are laser diodes.

8. Irradiation device according to any one of the preceding claims,
characterised in that
the luminous segments (1-4) respectively comprise a plurality of groups of uniformly arranged luminous means (6), the luminous means (6) within a group generating radiations in an essentially equal wavelength range and the individual groups of luminous means (6) generating radiations in different wavelength ranges.

9. Irradiation device according to any one of the preceding claims,
characterised in that
the individual luminous segments (1-4) generate directed radiation in the ultraviolet wavelength range.

10. Irradiation device according to any one of the preceding claims,
characterised in that
the individual luminous segments (1-4) generate directed radiation in the infrared wavelength range.

11. Irradiation device according to any one of the preceding claims,
characterised in that
the individual luminous segments (1-4) generate directed radiation in the visible wavelength range.

12. Irradiation device according to any one of the preceding claims,
characterised in that
a base surface of the individual luminous segments (1-4) is selected so that the radiations generated by the individual luminous segments (1-4) overlap essentially fully on a rectangular irradiation surface (A).

13. Irradiation device according to any one of claims 1-11,
characterised in that
a base surface of the individual luminous segments (1-4) is selected so that the radiations generated by the individual luminous segments (1-4) overlap essentially fully on a polygonal irradiation surface (A).

14. Irradiation device according to any one of claims 1-11,
characterised in that
a base surface of the individual luminous segments (1-4) is selected so that the radiations generated by the individual luminous segments (1-4) overlap essentially fully on a round irradiation surface (A).

15. Irradiation device according to any one of the preceding claims,
characterised in that
the individual luminous segment supports with the corresponding luminous segments (1-4) are arranged at an angle to one another and next to one another.

16. Irradiation device according to claim 15,
characterised in that
the luminous segment supports with the corresponding luminous segments (1-4) are arranged next to one another so that an essentially continuous emission surface is formed by the individual luminous segments (1-4).

17. Irradiation device according to any one of the preceding claims,
characterised in that
the luminous segment supports with the luminous segments (1-4) are fitted in a portable housing (5).

18. Irradiation device according to any one of the preceding claims,
characterised in that
the individual luminous segments (1-4) are arranged aligned with one another so that mid-axes of the individual luminous segments (1-4) extend in a common plane.

19. Irradiation device according to any one of the preceding claims,
characterised in that
the irradiation device comprises at least three luminous segments (1-4) arranged at an angle to one another.

20. Irradiation device according to any one of the preceding claims,
characterised in that
the irradiation device comprises a first group of luminous segments (1-3) arranged at an angle to one another and a second group of luminous segments (1-3) arranged at an angle to one another, the two groups of luminous segments (1-3) being arranged in a cross-sectional shape so that a particular luminous segment (1) is both part of the first group and part of the second group.

21. Irradiation device according to any one of the preceding claims,
characterised in that
the irradiation device comprises at least four luminous segments (1-4), the individual luminous segments (1-4) being arranged at an angle to one another, and in that the radiations of at least two luminous segments respectively overlap on the irradiation surface (A), so that each section of the irradiation surface (A) is irradiated by the overlapping radiation of at least two luminous segments.

22. Irradiation device according to any one of the preceding claims,
characterised in that
the irradiation device comprises at least five luminous segments (1-4) arranged at an angle to one another.

23. Irradiation device according to any one of the preceding claims,
characterised in that
the irradiation device comprises at least seven luminous segments (1-4) arranged at an angle to one another.

24. Irradiation device according to any one of the preceding claims,
characterised in that
the luminous segment supports (12) with the corresponding luminous segments (1-4) are arranged at an angle to one another, and in that the radiations of at least two luminous segments respectively overlap on the irradiation surface (A), so that each section of the irradiation surface (A) is irradiated by the overlapping radiation of at least two luminous segments, regions adjacent to the irradiation surface (A) being irradiated with a different irradiation intensity than the irradiation surface (A) by the radiations directed at them from the luminous segments (1-4).

25. Irradiation device according to claim 24,
characterised in that
the radiations generated by all the luminous segments (1-4) overlap essentially fully on the irradiation surface (A), whereas the regions adjacent to the irradiation surface (A) are irradiated by not all the luminous segments (1-4).

26. Irradiation device according to any one of the preceding claims,
characterised in that
the irradiation device comprises a detector unit (10, 11) for recording fluorescent radiation (C) stimulated on the irradiation surface (A) as a result of the irradiation by the radiations of the luminous segments (1-4).

27. Irradiation device according to claim 26,
characterised in that
the detector unit comprises an optical filter (11) with a transmission range corresponding to the stimulated fluorescent radiation (C).

28. Irradiation device according to claim 26 or 27,
characterised in that
the irradiation device is part of a medical photodynamic diagnosis system.

29. Irradiation device according to any one of the preceding claims,
characterised in that
the individual luminous segments are arranged essentially semicircularly in cross section.

30. Irradiation device according to claim 29,
characterised in that
the individual luminous segments (1-4) are arranged essentially hemicylindrically.

31. Irradiation device according to claim 30,
characterised in that
a plurality of groups of luminous segments (1-4) arranged at an angle to one another are arranged essentially parallel to one another, so that luminous segments (1-4) of the individual groups are arranged essentially hemicylindrically with the radiation being directed into the interior of the hemicylinder.

32. Irradiation device according to claim 31,
characterised in that
the individual luminous segment groups are directly adjacent to one another.

33. Irradiation system,
with two mutually opposite irradiation devices according to any one of claims 30-32, the irradiation surface (A) of the irradiation devices being arranged between the two irradiation devices.

34. Irradiation system according to claim 33,
characterised in that
the irradiation system is designed for whole body irradiation of a living being (P) to be positioned in the vicinity of the irradiation surface (A).

35. Irradiation system with an irradiation device according to any one of claims 1-32 for irraddiating a body section of a living being.

36. Irradiation system according to claim 35,
characterised in that
the irradiation device is fitted in a mobile housing (5) for positioning the irradiation device over the intended body section.

37. Irradiation system according to any one of claims 33-36,
characterised in that
the irradiation device is part of a medical photodynamic therapy system.

38. Irradiation system with an irradiation device according to any one of claims 1-32 for cosmetic treatment of a living being.

39. Irradiation system,
with an irradiation device according to any one of claims 1-32 for coupling the overlapping radiation striking the irradiation surface (A) into an optical waveguide (8).

40. Irradiation system with an irradiation device according to any one of claims 1-28,
the irradiation device comprising a multiplicity of luminous segments (1) which are arranged at an angle to one another, and in overall that they form an essentially circular cross-sectional shape, so that the radiations generated by the individual luminous segments (1) overlap in a central region of this circular cross-sectional shape.

41. Irradiation system according to claim 40,
characterised in that
the radiations generated by the individual luminous segments (1) of the irradiation device have a diameter which corresponds essentially to the diameter of the central region in which these radiations overlap.

42. Irradiation system according to claim 40,
characterised in that
the radiations generated by the individual luminous segments (1) of the irradiation device have a diameter which is less than the diameter of the central region in which the radiations overlap, so that each section of this central region is irradiated uniformly by the radiations of at least two luminous segments (1).

43. Irradiation system according to claim 42,
characterised in that
each section of the central region is irradiated by the radiations of at least three luminous segments (1) arranged next to one another.

44. Irradiation system according to any one of claims 40-43,
characterised in that
the irradiation system is designed for curing the surface of a medium (9) arranged in the central region of the irradiation system.

45. Irradiation system according to any one of claims 40-43,
characterised in that
the irradiation system is designed for irradiating a container (9) made of a radiation-transmissive material arranged in the central region of the irradiation system, the container containing biological material to be irradiated.
